(19) United States
(12) Patent Application Publication
(10) Pub. No.: US 2024/0207235 A1
(43) Pub. Date: Jun. 27, 2024

(54) NANOEMULSION FORMULATION FOR TREATING DISBETES MELLITUS (DM)

(71) Applicant: KING FAISAL UNIVERSITY, Al-Ahsa (SA)

(72) Inventors: TAMER MOHAMED SHEHATA, AL-AHSA (SA); MERVT MOHAMMED ALMOSTAFA, AL-AHSA (SA); HEBA S. ELSEWEDY, AL-AHSA (SA)

(21) Appl. No.: 18/087,646

(22) Filed: Dec. 22, 2022

(51) Int. Cl. A61K 31/426; A61K 9/107; A61K 47/10; A61K 47/26; A61K 47/44; A61P 3/08

(52) U.S. Cl. CPC A61K 31/426; A61K 9/1075; A61K 47/10; A61K 47/26; A61K 47/44; A61P 3/08

(57) ABSTRACT

The nanoemulsion (NE) formulation for treating diabetes mellitus (DM) includes pioglitazone (PGZ) and a nanoemulsion formulated with Nigella sativa oil (NSO). The (PGZ)-loaded nanoemulsion formulation can achieve a significant reduction in blood glucose levels when compared to commercially available pioglitazone formulations. Thus, it is believed that Nigella sativa oil (NSO) potentiates the hypoglycemic effect of pioglitazone (PGZ). In addition, the (PGZ)-loaded nanoemulsion formulation can be stably stored for a period of at least three months.

### BACKGROUND

### 1. Field

The disclosure of the present patent application relates to treatments for Diabetes Mellitus (DM) and, particularly, a nanoemulsion (NE) formulation for treating diabetes mellitus (DM), the nanoemulsion being loaded with Pioglitazone (PGZ)-.

### 2. Description of Related Art

Diabetes Mellitus (DM) is a metabolic disorder associated with an increased blood glucose level. In the past two decades the prevalence of DM and Type II diabetes, in particular, has increased to epidemic proportions worldwide. It is believed that chronically increased levels of blood glucose are a main reason for the development of diabetes complications, leading to a decreased life expectancy. This is mainly due to cardiovascular deaths, with a risk of coronary heart disease increased by two- to four-fold in this population.

Type II diabetes is characterized by insulin resistance. Pharmacological studies indicate that pioglitazone improves sensitivity to insulin in muscle and adipose tissue and inhibits hepatic gluconeogenesis. Pioglitazone improves glucose resistance while reducing circulating insulin levels and is useful in the treatment of diabetes, particularly type II diabetes.

Pioglitazone is currently marketed as ACTOS. Pioglitazone hydrochloride has the IUPAC name (RS)-5-(4-[2-(5-ethylpyridin-2-yl)ethoxy]benzyl)thiazolidine-2,4-dione monohydrochloride. (CAS Registry No. 111025-46-8). Pioglitazone has low solubility, which can make delivery of the drug challenging.

Thus, a nanoemulsion formulation for treating diabetes mellitus (DM) solving the aforementioned problems is desired.

### SUMMARY

The nanoemulsion (NE) formulation for treating diabetes mellitus includes pioglitazone (PGZ) and a nanoemulsion formulated with Nigella sativa oil (NSO). The (PGZ)-loaded nanoemulsion formulation can achieve a significant reduction in blood glucose levels when compared to commercially available pioglitazone formulations. Thus, it is believed that Nigella sativa oil (NSO) has a synergistic effect, potentiating the hypoglycemic effect of pioglitazone (PGZ). In addition, the (PGZ)-loaded nanoemulsion formulation can be stably stored for a period of at least three months.

These and other features of the present subject matter will become readily apparent upon further review of the following specification and drawings.

### BRIEF DESCRIPTION OF THE DRAWINGS

FIG. 1 is a chart showing the blood glucose level of rats induced with diabetes and treated with PGZ-loaded NE and blank NE, as compared to ACTOS® formulation and non-treated rats.

FIGS. 2A and 2B are linear correlation plots between predicted versus actual values representing the effect of the independent variables on (FIG. 2A) particle size R₁, and (FIG. 2B) the in vitro release study R₂, respectively.

FIGS. 3A and 3B depict (FIG. 3A) perturbation plots displaying the influence of each independent variable alone on (a) particle size R1 and (FIG. 3B) (b) in vitro release R2, respectively.

FIG. 4 is a plot showing particle size of the optimized PGZ-loaded NE formulation.

FIG. 5 is a plot illustrating in vitro release of the optimized PGZ-loaded NE formulation.

FIGS. 6A-6B are charts summarizing findings of a stability study of optimized PGZ-loaded NE following storage for 1 and 3 months at two different temperatures, 4±3° C. and 25±2° C., relative to (FIG. 6A) particle size and (FIG. 6B) in vitro release.

### DETAILED DESCRIPTION OF THE PREFERRED EMBODIMENTS

The following definitions are provided for the purpose of understanding the present subject matter and for construing the appended patent claims.

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It will be understood by those skilled in the art with respect to any chemical group containing one or more substituents that such groups are not intended to introduce any substitution or substitution patterns that are sterically impractical and/or physically infeasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals, such as, but not limited to, cattle, sheep, ferrets, swine, horses, poultry, rabbits, goats, dogs, cats and the like.

“Patient” as used herein refers to a subject in need of treatment of a condition, disorder, or disease, such as an acute or chronic airway disorder or disease.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The nanoemulsion (NE) formulation includes pioglitazone (PGZ) and a nanoemulsion formulated with Nigella sativa oil (NSO). The nanoemulsion formulation can have particle size of about 167 nm. The (PGZ)-loaded nanoemulsion formulation can achieve a significant reduction in blood glucose levels. In an embodiment, the in vitro release of the nanoemulsion formulation is about 89.5%. It is believed that Nigella sativa oil (NSO) potentiates the hypoglycemic effect of pioglitazone (PGZ).

A method of treating Diabetes Mellitus can include administering a therapeutically effective amount of the pioglitazone (PGZ)-loaded nanoemulsion (NE) to a subject in need thereof. The Diabetes Mellitus (DM) can be Type II Diabetes Mellitus (DM). A therapeutically effective amount of the pioglitazone (PGZ)-loaded nanoemulsion (NE) formulation or an amount effective to treat Diabetes Mellitus (DM) may be determined initially from the Examples described herein and adjusted for specific targeted diseases using routine methods. The present nanoemulsion (NE) formulation can be in unit dosage forms, such as tablets, pills, capsules, powders, or granules. The nanoemulsion (NE) formulation can be presented in a form suitable for daily, weekly, or monthly administration. The nanoemulsion (NE) formulation herein will contain, per dosage unit, e.g., tablet, capsule, and the like, an amount of the active ingredient necessary to deliver an effective dose.

The nanoemulsion (NE) formulation may be administered orally. Pioglitazone (PGZ) is a generally prescribed medication for managing type II diabetes. However, the low solubility of PGZ makes oral delivery of PGZ challenging. As described herein, when PGZ was incorporated into a nanoemulsion (NE) formulation prepared with Nigella sativa oil (NSO), PGZ activity was boosted in vivo.

The nanoemulsion can be formulated with Nigella sativa oil (NSO), a solubilizer, and a non-ionic surfactant, and, subsequently, loaded with PGZ. The solubilizer may be propylene glycol (PG). The non-ionic surfactant may be Tween 80 (polysorbate 80). The nanoemulsion may include, for example, from about 1 g to about 2 g of NSO, from about 0.25 g to about 0.75 g Tween 80, and from about 0.5 g to about 1.0 g propylene glycol (PG).

The nanoemulsion (NE) formulation has a suitable particle size and demonstrates optimal in vitro release. The nanoemulsion (NE) formulation may, for example, have a particle size of about 167.1 nm and in vitro release of about 89.5%. As described herein, a kinetic study revealed that the drug release followed the Korsmeyer-Peppas mechanism.

The NE formulation can remain stable for an extended period of time prior to use. For example, the NE formulation was found to be stable, showing non-significant variation in significant evaluated parameters when stored at 4° C. and 25° C. for a period of 3 months.

In vivo investigation of the nanoemulsion (NE) formulation showed a significant reduction in blood glucose level, which appeared to be enhanced by the presence of NSO.

The present subject matter will be better understood with reference to the following examples.

### Example 1

### Preparation of (PGZ)-Loaded Nanoemulsion (NE) Formulation

Two phases were prepared: an oily phase and an aqueous phase. For the oily phase, 30 mg of PGZ was mixed with 50 mg PEG-DSPE, and a specified amount of NSO and PG as a co-surfactant were added using a classic advanced vortex mixer (VELP Scintifica, Italy). An aqueous phase of up to 10 mL containing a specified amount of Tween 80 was also prepared. The two phases were mixed with homogenization for 10 min at 20,000 rpm using a high shear homogenizer (T 25 digital Ultra-Turrax, IKA, Staufen, Germany). A nanoemulsion loaded with PGZ was formed and subjected to 30 s sonication with a probe sonicator (XL-2000, Qsonica, Newtown, CT, USA) to obtain a homogenous NE.

The NE formulations were prepared based on the specified amounts of ingredients presented in Table 1.

TABLE 1
Responses of PGZ-loaded NE Formulations using BBD

| | Selected Factors | | | | |
|---|---|---|---|---|---|
| | NSO | Tween 80 | | Observed Responses | |
| Run | Concentration (g) | Concentration (g) | PG Concentration (g) | P. Size (nm) | In Vitro (%) |
| F1 | 1 | 0.25 | 0.75 | 181 ± 4.6 | 78.6 ± 3.8 |
| F2 | 1.5 | 0.5 | 0.75 | 188 ± 3. [symbol] | 74.1 ± 2.8 |
| F3 | 2 | 0.5 | 1 | 288 ± 4.6 | 54.0 ± 3.2 |
| F4 | 1.5 | 0.5 | 0.75 | 195 ± 5. [symbol] | 70.6 ± 2.2 |
| F5 | 1.5 | 0.75 | 0.5 | 211 ± 4.6 | 71.9 ± 3.1 |
| F6 | 1 | 0.75 | 0.75 | 166 ± 3.6 | 86.3 ± 2.9 |
| F7 | 2 | 0.25 | 0.75 | [symbol] 19 ± 5.4 | 48.2 ± [symbol] .1 |
| F8 | 1 | 0.5 | 1 | 170 ± 4.5 | 82.1 ± 3.4 |
| F9 | 1.5 | 0.75 | 1 | 203 ± 4.4 | 68.1 ± 3.6 |
| F10 | 1.5 | 0.5 | 0.75 | 190 ± 4. [symbol] | 72.1 ± 2.9 |
| F11 | 1 | [symbol] | 0.5 | 176 ± 4.6 | 80.3 ± 3.3 |
| F12 | 2 | 0.5 | 0.5 | 300 ± 5.3 | 51.5 ± 2.4 |
| F13 | 1.5 | 0.25 | 0.5 | 261 ± 4. [symbol] | 63.5 ± 3.1 |
| F14 | 2 | 0.75 | 0.75 | 270 ± 4.7 | 58.3 ± 2.6 |
| F15 | 1.5 | 0.25 | 1 | 246 ± 4. [symbol] | 66.5 ± 2.7 |

[symbol]

indicates data missing or illegible when filed

### Example 2

### NE Pharmacological Activity

Determination of the pharmacological hypoglycemic activity of the NE formulations was conducted by measuring the blood glucose level in diabetic rats following oral administration of the formulations. As shown in FIG. 1, blank NE showed significant reduction in blood glucose levels compared to the non-treated group at 2 and 4 h (p<0.05). The ACTOS®-treated group showed a maximum percentage blood glucose level reduction of 77.18% 4 h following drug administration. Interestingly, at the beginning of PGZ-loaded NE administration (1 h and 2 h), the drug produced faster blood glucose level reduction than the drug in tablet formulation (ACTOS®), which could be due to better dissolution rates of PGZ from the NE form compared to the tablet form. A maximum reduction in blood glucose level of 79.99% after 1 h of treatment was observed, followed by continuous hypoglycemic effects of the drug in NE form for 24 h. The NE treated group showed a rapid and significant reduction in PGZ at 1 h and 2 h following drug administration compared to all other formulations under investigation (p<0.05). The enhancement of the NE effect could be attributed to the synergistic effect of both PGZ drug and NSO during the first two hours of administration. The hypoglycemic in vivo results of PGZ with NSO, showing a similar or slightly enhanced effect compared to ACTOS®, indicates that this formulation can be recommended as an alternative dosage form for special hypoglycemic patients.

### Example 3

### Particle Size and In Vitro Release

The particle size of the prepared NE formulations was evaluated, and the results are displayed in Table 2.

TABLE 2
Experimental runs and the observed responses
of PGZ-loaded NE formulations using BBD.

| | Selected Factors | | | | |
|---|---|---|---|---|---|
| | NSO | Tween 80 | PG | Observed Responses | |
| | Concentration | Concentration | Concentration | P. Size | In vitro |
| Run | (g) | (g) | (g) | (nm) | (%) |
| F1 | 1 | 0.25 | 0.75 | 181 ± 4.6 | 78.6 ± 3.8 |
| F2 | 1.5 | 0.5 | 0.75 | 188 ± 3.0 | 74.1 ± 2.8 |
| F3 | 2 | 0.5 | 1 | 288 ± 4.6 | 54.0 ± 3.2 |
| F4 | 1.5 | 0.5 | 0.75 | 195 ± 5.0 | 70.6 ± 2.2 |
| F5 | 1.5 | 0.75 | 0.5 | 211 ± 4.6 | 71.9 ± 3.1 |
| F6 | 1 | 0.75 | 0.75 | 166 ± 3.6 | 86.3 ± 2.9 |
| F7 | 2 | 0.25 | 0.75 | 319 ± 5.4 | 48.2 ± 3.1 |
| F8 | 1 | 0.5 | 1 | 170 ± 4.5 | 82.1 ± 3.4 |
| F9 | 1.5 | 0.75 | 1 | 203 ± 4.4 | 68.1 ± 3.6 |
| F10 | 1.5 | 0.5 | 0.75 | 190 ± 4.0 | 72.1 ± 2.9 |
| F11 | 1 | 0.5 | 0.5 | 176 ± 4.6 | 80.3 ± 3.3 |
| F12 | 2 | 0.5 | 0.5 | 300 ± 5.3 | 51.5 ± 2.4 |
| F13 | 1.5 | 0.25 | 0.5 | 261 ± 4.5 | 63.5 ± 3.1 |
| F14 | 2 | 0.75 | 0.75 | 270 ± 4.7 | 58.3 ± 2.6 |
| F15 | 1.5 | 0.25 | 1 | 246 ± 4.9 | 66.5 ± 2.7 |

It was observed that the particle size was influenced by the different independent variables A, B and C. The particle sizes of all the NE formulations ranged from 166±3.6 up to 319±5.4 nm. It was observed that increasing concentration of NSO from 1 g to 2 g was associated with a parallel increase in NE particle size, possibly due to increase in the dispersed phase. Another interpretation is that by increasing the oil concentration, disruption of the particles was reduced, and hence the rate of collision could increase, leading to coalescence and aggregation of droplets. However, the particle size was influenced by the surfactant and co-surfactant concentration using the same NSO concentration. Using 1 g NSO, particle size was small when using 0.75 g of Tween 80 and 0.75 g of PG (166 nm), while it was larger (181 nm) when using 0.25 g Tween 80 and 0.75 g PG. It was concluded that higher surfactant and co-surfactant concentration led to lowering in particle size of the formulated NE. These findings can be interpreted based on lowered interfacial tension with increasing surfactant concentration and consequent decrease in the particle size of the NE. The previous data was confirmed with the following mathematical equation:

$$R 1 = 191 + 60.5 * A - 19.625 * B - 5.125 * C - 8.5 * AB - 1.5 * AC + 1.75 * BC + 23.125 * A 2 + 19.875 * B 2 + 19.375 * C 2 \qquad ( 1 )$$

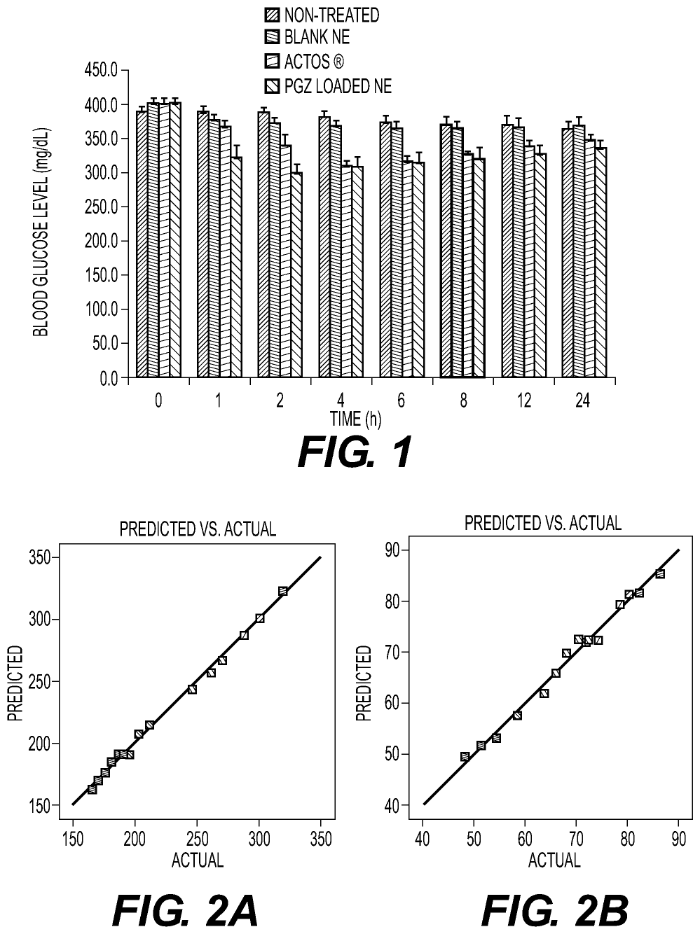

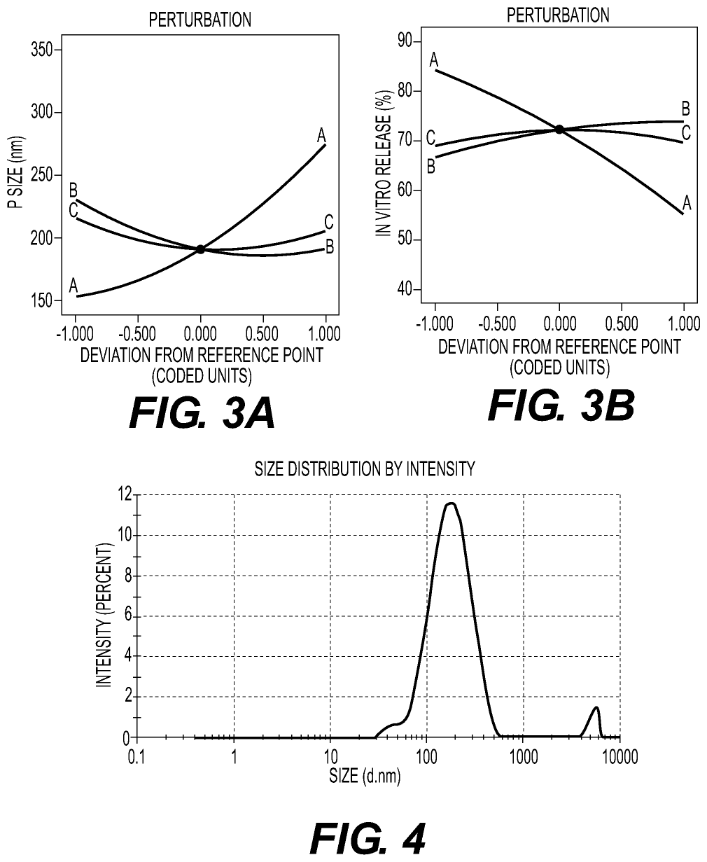

The positive sign of NSO (A) indicates a parallel influence. However, the negative sign for the B and C factors indicates that there was an inverse relation between these factors and the response R1. Further, the response surface plot confirmed the previous relation between the three factors and the response R1. Additionally, Table 3 and FIGS. 2A-2B illustrate a linear correlation between the predicted and actual data. This was confirmed by the value of the adjusted (0.9899) and predicted R2 (0.9519), with reasonable agreement between them. Moreover, the perturbation plots, shown in FIGS. 3A-3B, were generated to clarify the effect of each factor on the selected response if all other factors were kept constant. It was apparent that factor A had a more noticeable effect on R1 than the other two factors, B and C, with the curvature indicating the sensitivity. Additionally, the direction of the perturbation plot confirmed that factor A had a synergistic effect on particle size R1, while factors B and C had antagonistic effects.

TABLE 3
Statistical Analysis and Fit Statistics
of all Dependent Variables R₁ and R₂.

| | R₁ | | R₂ | |
|---|---|---|---|---|
| Source | F-Value | p-Value | F-Value | p-Value |
| Model | 153.39 | <0.0001 | 58.36 | 0.0002 |
| A | 1090.58 | <0.0001* | 476.08 | <0.0001* |
| B | 114.75 | 0.0001* | 28.43 | 0.0031* |
| C | 7.83 | 0.0381* | 0.4349 | 0.5387 |
| AB | 10.76 | 0.0219* | 0.4090 | 0.5506 |
| AC | 0.3352 | 0.5877 | 0.1022 | 0.7621 |
| BC | 0.4562 | 0.5294 | 2.82 | 0.1540 |
| A² | 73.54 | 0.0004* | 6.40 | 0.0525 |
| B² | 54.32 | 0.0007* | 3.97 | 0.1029 |
| C² | 51.62 | 0.0008* | 9.10 | 0.0295* |
| Lack of Fit | 2.76 | 0.2759 | 1.24 | 0.4763 |

| | | |
|---|---|---|
| R² | 0.9964 | 0.9906 |
| Adjusted R² | 0.9899 | 0.9736 |
| Predicted R² | 0.9519 | 0.8945 |
| Adequate Precision | 37.8766 | 23.5140 |
| Model | Quadratic | Quadratic |
| Remark | Suggested | Suggested |

A, NSO concentration (g);
B, Tween 80 concentration (g);
C, PG concentration (g);
R₁, particle size (nm); and
R₂, in vitro release (%);
*significant, p < 0.05.

### Example 4

### Influence of the Independent Variables on R2

An in vitro release study was conducted for all PGZ-loaded NE formulations with release values ranging between 48.2±3.1 and 86.3±2.9% after 12 h, as shown in Table 1. It was clear that NSO indirectly affected the percentage of PGZ released from the NE formulation, with increasing NSO resulting in a lower R2 percentage. This might be attributed to the larger particle size, since increasing oil concentration would cause a relative increase in NE particle size, providing a small surface area, lowering the percentage release from the formulation. However, with a constant concentration of NSO, it was noted that increasing surfactant (B) and co-surfactant (C) concentrations enhance PGZ release from the developed NE. This may have been a result of the surfactant lowering the interfacial tension between the formulation and the surrounding aqueous media of release. This would allow for more wetting of the drug, facilitating penetration, which would increase the rate of drug released from the formulation. The obtained mathematical equation emphasized the earlier noticed facts where factor A had a negative sign indicating an opposed effect, in contrast to the other two factors, B and C that had a positive sign, indicating synergistic action:

$$R 2 = 72.2667 - 14.475 * A + 3.5375 * B + 0.4375 * C + 0.6 * AB + 0.3 * AC - 1.575 * BC - 2.47083 * A 2 - 1.94583 * B 2 - 2.94583 * C 2 \qquad ( 2 )$$

For further confirmation, BBD software was used to generate certain graphs to highlight the relation between the selected independent variables and the examined in vitro release response R2. An integrated relation between the factors and response R2 were determined. FIG. 2B shows the linear correlation between the A, B and C independent variables and the observed R2. Additionally, the values of the adjusted and predicted R2 of 0.9736 and 0.8945, respectively, were closely aligned with each other and were in reasonable agreement, as shown in FIG. 2B. The perturbation graph displayed in FIG. 3B strongly supports the above findings. It was observed that the direction of factor A indicated an inverse relation with the response R2. The other factors, B and C, were less prominent than factor A. However, they evidenced a synergistic effect on R2.

### Example 5

### PGZ-loaded NE Morphology, Kinetic Activity and Stability

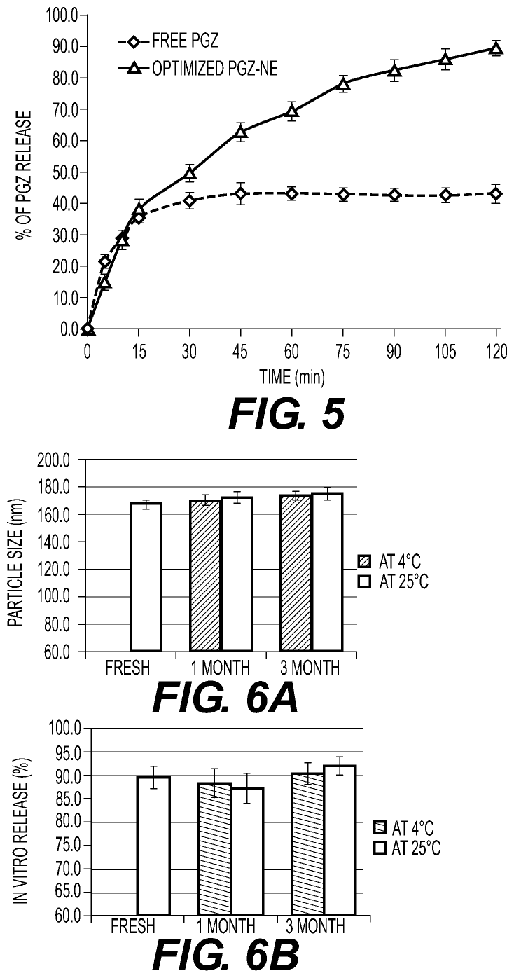

The optimized formula was developed and evaluated for a particle size of 167.1±3.43 nm with PDI of 0.308, as shown in FIG. 4. The data indicated that the NE was homogenous and that the particles were distributed in a narrow range of sizes, which is a good indicator of formulation stability. The in vitro release of PGZ from the optimized formula was compared to the release of PGZ from the free drug. As shown in FIG. 5, it was notable that the PGZ dissolution was very poor. It demonstrated maximum release 45 min. after the start of the experiment with a release value of 43.27±3.6%. This could be attributed to the crystalline nature of the drug in addition to its poor solubility in the release media. The optimized PGZ-loaded NE showed an increase in the rate of dissolution of 89.5±2.4% after 120 min. This was ascribed to the small particle size of the formulation that would provide a larger surface area contributing to enhanced release. Additionally, it is well-known that the presence of a surfactant and co-surfactant would enhance the dissolution of the drug in the release media. These factor suggest that PGZ release would be significantly enhanced within the NE formulation.

Application of different kinetic models was used to determine the mechanism by which the drug would diffuse from the formula during in vitro release. On constructing different models representing the kinetics of drug release, it was noted that the Korsmeyer and Peppas model provided the best mechanism for PGZ release from the NE. It showed a linear correlation and displayed the highest value for the correlation coefficient (R2), being very close to 1 (0.9789). The permeability exponent (n) for the PGZ-loaded NE formulation was found to be 1.0292, which was higher than 0.89. This indicates that the release from the NE system followed non-Fickiansupercase II transport diffusion.

### Example 6

### Diabetically Induced Rats

Animals in the current study were induced to be diabetic using Streptozotocin (60 mg/kg) via intraperitoneal injection. Streptozotocin has the capability to induce hyperglycemia within 3 days following injection since it destroys beta cells. The animals were tested for their blood glucose level after fasting using a digital One Touch® Ultra 2® glucometer. Once the blood glucose level reached 300 mg/dl, the animals were considered diabetic and were prepared for the experimental study.

Three days following induction of hyperglycemia, diabetic rats were divided into four groups, each having six animals. The animals received the following treatment orally using oral gavage: Group I: negative control group received saline orally (non-treated); Group II: positive control group received marketed PGZ product (ACTOS ®) (equivalent to 30 mg/kg); Group III: rats treated with NE free from PGZ (blank NE); Group IV: rats treated with optimized PGZ-loaded NE (equivalent to 30 mg/kg).

Starting from time 0 h up to 24 h, blood samples were taken from the animals' tails and were applied to a glucometer strip to be analyzed for blood glucose level using a digital One Touch® Ultra 2® glucometer. The animals were fasted during 12 h of the study and fed later. The results are discussed in Example 1.

It is to be understood that the nanoemulsion formulation for treating Diabetes Mellitus (DM) is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A nanoemulsion (NE) formulation, consisting of:
pioglitazone; and
a nanoemulsion, the pioglitazone being loaded therein, the nanoemulsion consisting of Nigella sativa oil (NSO), propylene glycol (PG), and polysorbate 80;
wherein the NSO and the PG are present in the nanoemulsion in a ratio of NSO:PG of 1:0.75 and the NSO and the polysorbate 80 are present in the nanoemulsion in a ratio of NSO: polysorbate 80 of 1:0.75; and wherein the nanoemulsion (NE) formulation has a particle size of about 167 nm.

2-8. (canceled)

9. A method for treating Type II Diabetes Mellitus, comprising administering a therapeutically effective amount of the nanoemulsion (NE) formulation of claim 1 to a patient in need thereof.

10. A method for treating Type II Diabetes Mellitus, comprising administering a therapeutically effective amount of a pioglitazone (PGZ)-loaded nanoemulsion (NE) formulation to a patient in need thereof, the pioglitazone (PGZ)-loaded nanoemulsion (NE) formulation consisting of:
pioglitazone; and
a nanoemulsion including the pioglitazone loaded therein, the nanoemulsion consisting of Nigella sativa oil (NSO), propylene glycol (PG), and polysorbate 80;
wherein the NSO and the PG are present in the nanoemulsion in a ratio of NSO:PG of 1:0.75, and the NSO and the polysorbate 80 are present in the nanoemulsion in a ratio of NSO:polysorbate 80 of 1:0.75; and wherein the pioglitazone (PGZ)-loaded nanoemulsion (NE) formulation has a particle size of about 167 nm.

11-20. (canceled)
